(19) United States
(12) Patent Application Publication
(10) Pub. No.: US 2017/0203118 A1
(43) Pub. Date: Jul. 20, 2017

(54) BEAUTY APPARATUS, AND WORKING TIP, HANDLE AND HAND TOOL THEREOF

(71) Applicant: Beijing Icandemy Tech Co., Ltd., Beijing (CN)

(72) Inventor: Peng Chang, Beijing (CN)

(21) Appl. No.: 15/405,437

(22) Filed: Jan. 13, 2017

(30) Foreign Application Priority Data
Jan. 14, 2016 (CN) 201610023868.2

(51) Int. Cl. A61N 5/02; A61N 7/00

(52) U.S. Cl. CPC A61N 5/022; A61N 7/00; A61N 2007/0034

(57) ABSTRACT

The present invention discloses a beauty apparatus, and a working tip, a handle and a hand tool thereof. Arrays, combined by magnetically-controlled tips and magnetically-controlled sensors, are correspondingly provided on the working tip and the handle of the beauty apparatus. When the working tip is mounted on the beauty apparatus, automatic detection for the type of a working tip is achieved by using the magnetically-controlled tips and the magnetically-controlled sensors arranged on the working tip and the handle, and according to the result of automatic detection, a corresponding working mode is automatically invoked without requiring any manual setting by an operator. Thus, the usage is faster and more convenient. Furthermore, it can avoid the occurrence of unsatisfactory cosmetic results or even medical accidents because the operator forgets setting or sets improperly in an existing manual setting mode, and improve the degree of user satisfaction.

### CROSS REFERENCE TO RELATED APPLICATION

This application claims priority to and takes the benefit of Chinese Patent application no. 201610023868.2 filed on Jan. 14, 2016, the contents of which are herein incorporated by reference.

### TECHNICAL FIELD

The present invention relates to the field of beauty apparatuses, and specifically relates to a beauty apparatus, and a working tip, a handle and a hand tool thereof.

### BACKGROUND OF THE PRESENT INVENTION

With the increasing improvement of living standards, people have higher and higher pursuits for spirit and material and even more urgent pursuits for beauty. As the saying goes, “Clothes make the man”, the skin is the most primitive “clothes” for people. Thus, people pay more and more attention to maintenance and care of the skin. A variety of beauty apparatuses for the purpose of maintenance of the skin emerge For example, beauty apparatuses, such as radio-frequency beauty apparatuses and ultrasonic beauty apparatuses which are the most widely used at present, are used for treating and improving the skin in various body parts, such as face, arms and back. However, the skin tissues, skin distribution and energy absorption at different body parts are different. Accordingly, for the treatment on different body parts, the shape of a used working tip of the beauty apparatus is required to match with the corresponding parts. For example, the shape of a portion of the working tip of the beauty apparatus in contact with the skin during a face treatment is different from that during a back treatment. Moreover, the required amount of energy is also different due to the difference in the skin tissues at various body parts. In order to meet the above requirements, at present, a concept of “one device with multiple tips” has been proposed for a variety of beauty apparatuses. In other words, a beauty apparatus host is equipped with various types of replaceable working tips of the beauty apparatus, and multiple working modes having different energy outputs can also be provided for different types of the working tips of the beauty apparatus. The following description will be given by taking a radio-frequency beauty apparatus as an example.

Studies have proven that the activity of cell division is very slow in a woundless basal layer of the skin. Once the skin is injured, the division rate of basal cells in the epidermis about 2 mm around this wound is more than 40 times faster than usual. By utilizing the principle that the radio-frequency waves can enable cell molecules to have strongly resonant rotation (tens of millions of times per second) to generate heat energy while directly penetrating through the skin to form impedance by the skin, so as to rise the local temperature of the skin, cause a “pseudo wound” on the skin, instantly tighten the collagen and stimulate regeneration of the collagen through the dermal stimulation, the radio-frequency beauty apparatus has the functions of redraping and tightening the skin, eliminating wrinkles and slimming the face. After the treatment, the collagen will be gradually proliferated and reformed, so as to redrape and tighten the sagging skin. Radio-frequency totally surpasses and replaces laser and IPL (intense pulsed light and photon) in many aspects. The radio-frequency technology has amazing effects in the aspects of filling wrinkles, eliminating scars, restoring elasticity and luster of the skin, dissolving fat, slimming, etc.

However, at present, for a variety of beauty apparatuses in which the radio-frequency technology is dominant, the energy injection process is manually adjusted and controlled by an operator. For example, if what is used currently is a working tip for face treatment used to carry out treatment to the face, the operator is required to manually and correspondingly adjust the output energy after the replacement of the working tip; if the output energy of the beauty apparatus is too large due to improper adjustment or forgetting to adjust, it will cause heavy and unrecoverable injury to the skin; and if the output energy of the beauty apparatus is too small, it is unable to cause pseudo injury to the skin tissues, and as a result, the cosmetic result will be affected. Furthermore, since the degrees of energy absorption at different body parts are also different, if the energy output is controlled improperly, it will not only affect the cosmetic result, but sometimes even cause medical accidents. Therefore, for the existing “one device with multi-tip” beauty apparatuses, when the working tips of different types are replaced and used, the operator is required to manually adjust a corresponding working mode according to the type of the currently used working tip, resulting in very high requirements on the operator and high hazards. The users, especially non-professional users, will have low degree of satisfaction in aspects of safety and cosmetic result.

### SUMMARY OF THE PRESENT INVENTION

A main technical problem to be solved by the present invention is to provide a beauty apparatus, and a working tip, a handle and a hand tool thereof, to solve the problems that a corresponding working mode (including corresponding features, working parameters, protection parameters, etc., which are collectively called herein working mode changes) is to be manually adjusted during the replacement of a working tip of a different type in an existing beauty apparatus.

In order to overcome the above technical problems, the present invention provides a working tip of a beauty apparatus, including: a working tip body, and a treating tip and a group of magnetically-controlled tips respectively provided at front and rear ends of the working tip body, the group of magnetically-controlled tips including at least one magnetically-controlled tip; and

when in use, the rear end of the working tip body is assembled and connected to a front end of a handle, and when assembled well, the magnetically-controlled tips in the group of magnetically-controlled tips are respectively coordinated with corresponding magnetically-controlled sensors at the front end of the handle so as to trigger the corresponding magnetically-controlled sensors by way of a magnetic field to generate an electric identification signal.

In order to overcome the above technical problems, the present invention further provides a handle of a beauty apparatus, including a handle body, and a magnetically-controlled sensor array having at least two magnetically-controlled sensors which is provided at a front end of the handle body, the magnetically-controlled sensors being connected to a beauty apparatus host; and

when in use, the front end of the handle body is assembled and connected to a rear end of a working tip, and when assembled well, the magnetically-controlled sensors are respectively coordinated with corresponding magnetically-controlled tips in a group of magnetically-controlled tips provided at the rear end of the working tip to generate an electric identification signal and then transmit the electric identification signal to the beauty apparatus host.

In order to overcome the above technical problems, the present invention further provides a hand tool of a beauty apparatus, including a handle and at least two working tips of different types; the handle includes a handle body, and a magnetically-controlled sensor array having at least two magnetically-controlled sensors which is provided at a front end of the handle body, the magnetically-controlled sensors being connected to a beauty apparatus host; the working tip includes a working tip body, and a treating tip and a group of magnetically-controlled tips respectively provided at the front and rear ends of the working tip body, the group of magnetically-controlled tips including at least one magnetically-controlled tip, and combinations of the magnetically-controlled tips in the group of magnetically-controlled tips are different in the working tips of different types; and

when in use, the rear end of the selected working tip body is assembled and connected to the front end of the handle, and when assembled well, the magnetically-controlled tips in the group of magnetically-controlled tips at the rear end of the working tip body are respectively coordinated with corresponding magnetically-controlled sensors at the front end of the handle so as to trigger the corresponding magnetically-controlled sensors to generate an electric identification signal and then transmit the electric identification signal to the beauty apparatus host.

In order to overcome the above technical problems, the present invention further provides a beauty apparatus, including a beauty apparatus host and the hand tool as described above; and the beauty apparatus host is provided with a controller;

when in use, the rear end of the selected working tip body is assembled and connected to the front end of the handle, and when assembled well, the magnetically-controlled tips in the group of magnetically-controlled tips at the rear end of the working tip body are respectively coordinated with corresponding magnetically-controlled sensors at the front end of the handle so as to trigger the corresponding magnetically-controlled sensors to generate an electric identification signal and then transmit the electric identification signal to the beauty apparatus host; and

the type of a working tip currently mounted on the handle is identified by the controller according to the electric identification signal and a type correspondence list, and a working mode corresponding to the type of the working tip of the beauty apparatus is set according to the result of identification.

In one embodiment of the present invention, the combinations of the magnetically-controlled tips in the group of magnetically-controlled tips are different in the working tips of different types, including: the number of the magnetically-controlled tips is different, and/or the position order for arranging the magnetically-controlled tips are different.

In one embodiment of the present invention, the type correspondence list includes a correspondence between various combinations of the magnetically-controlled tips and the type of the working tips; identifying the type of a working tip currently mounted on the hand tool of the beauty apparatus by the controller includes:

obtaining, by the controller, a combination of magnetically-controlled tips in the group of magnetically-controlled tips on the working tip, according to the received electric identification signal; and

selecting, by the controller and from the type correspondence list, the identified type of the working tip corresponding to the combination of the magnetically-controlled tips.

In one embodiment of the present invention, the controller is also configured to control the activation of the beauty apparatus host according to the working mode, and then stop the beauty apparatus host when a magnetically-controlled sensor inputting the electric identification signal is detected to input a signal having a level opposite to that of the electric identification signal.

In one embodiment of the present invention, the beauty apparatus host is a radio-frequency host, and the hand tool is a hand tool of a radio-frequency beauty apparatus.

In one embodiment of the present invention, the beauty apparatus further includes a human-computer interaction console connected to the beauty apparatus host, configured to issue at least one of a working mode setting instruction and a working state control instruction to the beauty apparatus host, and/or display information from the beauty apparatus host.

In one embodiment of the present invention, the magnetically-controlled sensor is a proximity magnetically-controlled switch.

In one embodiment of the present invention, the proximity magnetically-controlled switch is a reed switch or a Hall sensor.

In one embodiment of the present invention, when the proximity magnetically-controlled switch is a Hall sensor, the Hall sensor includes a Hall element and a magnet adjacent to the Hall element, and the magnetically-controlled tips are made of magnetic conducting material.

In one embodiment of the present invention, when the proximity magnetically-controlled switch is a reed switch, the reed switch is a normally-closed reed switch or normally-open reed switch, and the magnetically-controlled tips contain magnetic material.

In one embodiment of the present invention, when the reed switch is a normally-closed reed switch, the electric identification signal is a low-level signal; and when the reed switch is a normally-open reed switch, the electric identification signal is a high-level signal.

The present invention has the following beneficial effects.

The present invention provides a beauty apparatus, and a working tip, a handle and a hand tool thereof, including a handle and at least two working tips of different types; a group of magnetically-controlled tips are provided at a rear end of each type of the working tip body, and the combinations of the magnetically-controlled tips in the group of magnetically-controlled tips are different in the working tips of different types; and a magnetically-controlled sensor array, having at least two magnetically-controlled sensors, is provided at a front end of the handle. When in use, the rear end of the currently selected working tip body is assembled and connected to the front end of the handle, and when assembled well, the magnetically-controlled tips in the group of magnetically-controlled tips at the rear end of the working tip body are respectively coordinated with corresponding magnetically-controlled sensors at the front end of the handle so as to trigger the corresponding magnetically-controlled sensors to generate an electric identification signal and then transmit the electric identification signal to the beauty apparatus host. The type of a working tip currently mounted on the handle of the beauty apparatus is identified by the controller of the beauty apparatus host according to the received electric identification signal and the preset type correspondence list, so that a working mode corresponding to the type of the working tip of the beauty apparatus is automatically set according to the result of identification. Thus it can be seen that, when a working tip is mounted on the beauty apparatus, automatic detection for the type of a working tip is achieved by using the magnetically-controlled tips and the magnetically-controlled sensors arranged on the working tip and the handle, and according to the result of automatic detection, a corresponding working mode is automatically invoked without requiring any manual setting by an operator. Thus, the usage is faster and more convenient. Furthermore, it can avoid the occurrence of unsatisfactory cosmetic results or even medical accidents because the operator forgets setting or sets improperly in an existing manual setting mode, and improve the degree of user satisfaction. Moreover, it can greatly lower the professional requirements on the operator, and is more conducive to product promotion.

Further, the magnetically-controlled sensor in the present invention can specifically be a proximity magnetically-controlled switch. Because it enables detection in a non-contact manner, no abrasion and damage will occur. The radio-frequency beauty apparatus generally has an operating frequency of 1 Mhz to 40 Mhz, an output voltage peak value of 100 Vpp to 400 Vpp and an output power of 10 W to 300 W. By identifying a working tip by a magnetic field, the reliability at a high frequency and under a high voltage is well ensured. A working tip can be identified by placing only several magnetically-controlled tips on the working tip, with no elements such as electronic elements and cells need to be mounted. During a beauty treatment operation, the working tip contacts the skin very frequently, and skin care liquids will also be used. Since those skin care liquids will not degrade the identification function of the working tip, good practicality is ensured. Meanwhile, compared with the output control by mechanical motion, a non-contact output mode is employed, so that it has the advantages of fast response, longer service life and no influence on the life of contacts, and can guarantee fast and reliable detection.

### BRIEF DESCRIPTION OF THE DRAWINGS

FIG. 1 is a structure diagram of a beauty apparatus according to an embodiment of the present invention;

FIG. 2 is a structure diagram of a hand tool of the beauty apparatus according to the embodiment of the present invention;

FIG. 3 is a structure diagram of a beauty apparatus host according to the embodiment of the present invention;

FIG. 4 is a structure diagram of a working tip of the beauty apparatus according to the embodiment of the present invention;

FIG. 5 is a structure diagram of a handle tip of the beauty apparatus to according to the embodiment of the present invention;

FIG. 6 is a diagram showing the combination of the working tip of FIG. 4 and the handle of FIG. 5;

FIG. 7 is a structure diagram of a beauty apparatus having a human-computer interaction console according to the embodiment of the present invention;

FIG. 8 is a structure diagram of a Hall sensor according to the embodiment of the present invention;

FIG. 9 is a structure diagram of a normally-closed reed switch according to the embodiment of the present invention;

FIG. 10 is a schematic diagram of the normally-closed reed switch according to the embodiment of the present invention, when in use;

FIG. 11 is a structure diagram of a normally-open reed switch according to the embodiment of the present invention; and

FIG. 12 is a schematic diagram of the normally-open reed switch according to the embodiment of the present invention, when in use.

### DETAILED DESCRIPTION OF THE PRESENT INVENTION

In the present invention, automatic detection for the type of a working tip is achieved by using magnetically-controlled tips and magnetically-controlled sensors arranged on a working tip and a handle, and according to the result of automatic detection, a corresponding working mode can be automatically invoked without requiring any manual setting by an operator. Thus, the usage is faster and more convenient. Furthermore, it can avoid the occurrence of unsatisfactory cosmetic results or even medical accidents because the operator forgets setting or sets improperly in an existing manual setting mode. This technique is applicable to various beauty apparatuses having a demand of replacing a working tip, such as an ultrasonic beauty apparatus, and especially applicable to a radio-frequency beauty apparatus. The present invention will be further described in detail with reference to the accompanying drawings by specific implementations.

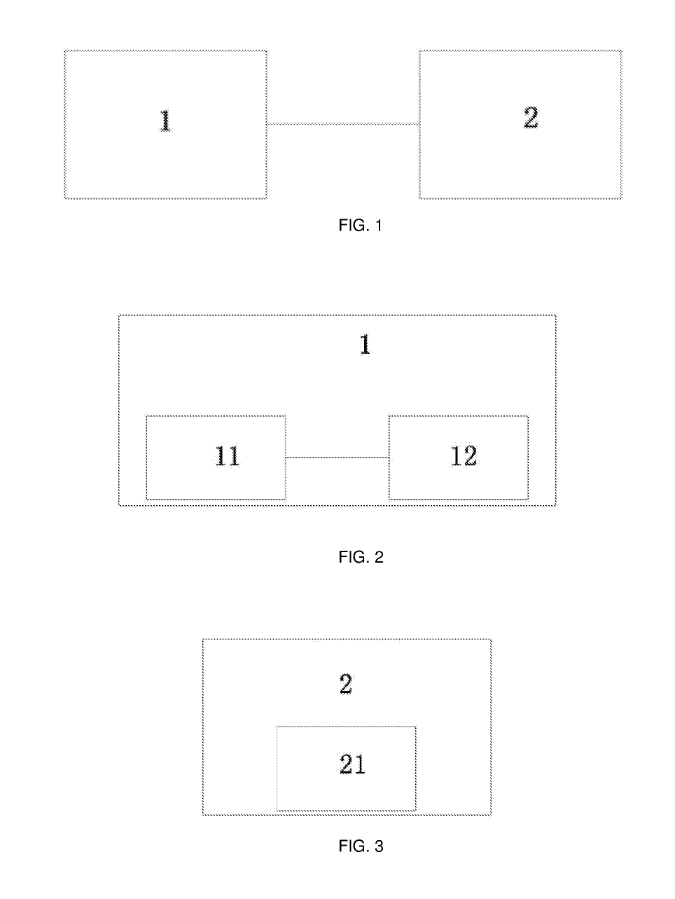

With reference to FIG. 1, a beauty apparatus according to this embodiment includes a hand tool 1 and a beauty apparatus host 2.

Wherein, with reference to FIG. 2, the hand tool 1 of the beauty apparatus includes a working tip 11 and a handle 12. There are various types of the working tips 11 suitable for different body parts and/or different ages and/or different genders and/or different skin types (for example, white skin, black skin and yellow skin). A group of magnetically-controlled tips, comprising at least one magnetically-controlled tip, are provided on the working tip 11, and combinations of the magnetically-controlled tips in the group of magnetically-controlled tips are different in the working tips 11 of different types. A magnetically-controlled sensor array having at least two magnetically-controlled sensors, which is coordinated with the group of magnetically-controlled tips, is provided on the handle 12.

With reference to FIG. 3, the beauty apparatus host 2 includes a controller 21. The controller 21 can be a control circuit of the beauty apparatus host 2 and can be configured to analyze signals, make various control policies, issue control instructions, and the like. It should be understood that, the beauty apparatus host 2 in this embodiment further includes an energy generation module and various accompanying modules. For example, a radio-frequency beauty apparatus host includes at least one of a radio-frequency boosting module, a power management module, a voltage regulator module, a denoising module and a circuit protection module. The controller 21 in this embodiment is electrically connected to the magnetically-controlled sensors, to receive electric identification signals from the magnetically-controlled sensors.

When in use, the currently used working tip 11 is assembled and connected to the handle 12, and when assembled well, the magnetically-controlled tips in the group of magnetically-controlled tips on the working tip 11 are respectively coordinated with corresponding magnetically-controlled sensors at the front end of the handle 12 so as to trigger the corresponding magnetically-controlled sensors to generate an electric identification signal and then transmit the electric identification signal to the controller 21. The type of a working tip currently mounted on the handle 12 of the beauty apparatus is identified by the controller 21 according to the electric identification signal and a preset type correspondence list, and a working mode corresponding to the type of the working tip of the beauty apparatus is set according to the result of identification. In this embodiment, each working mode corresponds to a group of working parameters and protection parameters, and the protection parameters can be different or the same in different working modes.

It should be understood that, in this embodiment, both of the shapes of the working tip 11 and the handle 12 and the connection mode for assembling the two can be set according to specific needs, and the structure, shape and connection mode in different design schemes can vary. For example, the shape of a cross section of the working tip 11 can be set as a circle, an ellipse, etc., and the shape of the handle can be set as various shapes which are convenient for a user to hold, such as a long bar; and the working tip 11 and the handle 12 can be preferably connected detachably, for example, in various clamping ways.

In addition, in this embodiment, due to factors such as different types of beauty apparatuses or different energy power requirements, the beauty apparatus host 2 can be independent of the handle 12, or provided in the handle 12 directly when its shape and volume are small enough to be physically integrated with the handle 12.

As mentioned above, in this embodiment, the working tips 11 can be classified into many types, according to at least one of different body parts, different ages, different genders and different skin types. For example, the working tips can be classified into working tips of different types respectively suitable for the face, neck, arms and other parts, according to different body parts. In this embodiment, treating tips for working tips of different types are generally different.

In order to better understand the solutions of the present invention, the components of the beauty apparatus according to the present invention will be described by several examples.

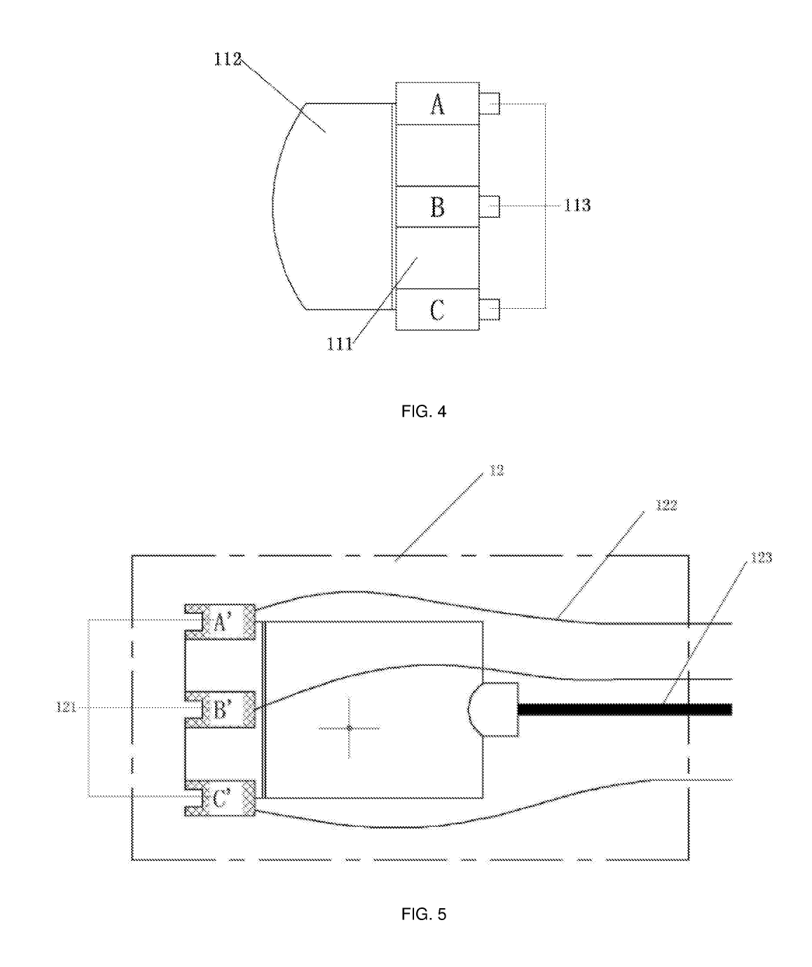

With reference to FIG. 4, a working tip 11 of the beauty apparatus according to one embodiment of the present invention is shown, including a working tip body 111, a treating tip 112 provided at a front end of the working tip body 111, and a group of magnetically-controlled tips 113 respectively provided at a rear end of the working tip body 111. As mentioned above, the specific shape and type of the treating tip 112 of FIG. 4 can be flexibly set according to the requirements of the beauty apparatuses of different types. In this embodiment, combinations of the magnetically-controlled tips in the group of magnetically-controlled tips 113 are different in the working tips 111 of different types. In this embodiment, the combinations of the magnetically-controlled tips include but are not limited to the following combinations.

First combination: a number of magnetically-controlled tips are combined, i.e., the number of magnetically-controlled tips in the group of magnetically-controlled tips on the working tips of different types is different, so that the number of eventually generated electric identification signals is different.

Second combination: the position order for arranging the magnetically-controlled tips are different, i.e., the number of magnetically-controlled tips in the group of magnetically-controlled tips on the working tips of different types can be the same, the position of at least one of the magnetically-controlled tips in a different group of magnetically-controlled tips is different from that in other groups.

Third combination: the first combination and the second combination are used together.

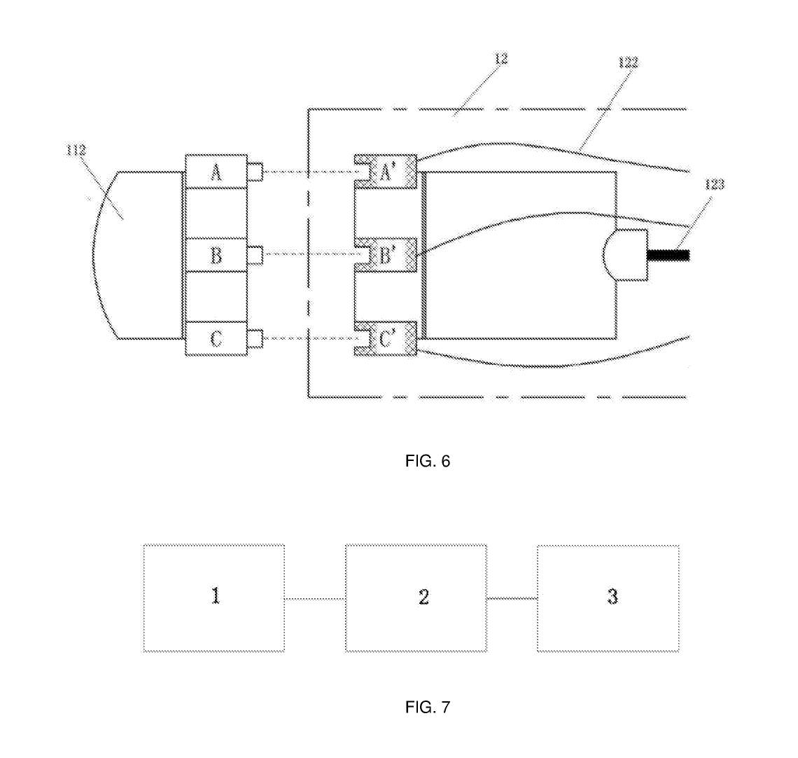

With reference to FIG. 5, a handle 12 of the beauty apparatus according to the embodiment of the present invention is shown. A magnetically-controlled sensor array 121 is provided at a front end of the handle 12, and magnetically-controlled sensors in the magnetically-controlled sensor array 121 are respectively connected to the controller 21 in the beauty apparatus host 2 through an identification signal transmission cable 122; and the handle 12 is also connected to the energy generation module in the beauty apparatus host 2 through an energy output cable 123. In FIG. 5, there are three magnetically-controlled sensors in the magnetically-controlled sensor array 121 at the front end of the handle 12. It should be understood that the number of magnetically-controlled sensors in the magnetically-controlled sensor array 121 can be set according to the number of the types of the working tips and the specific combination of the magnetically-controlled tips. With reference to FIG. 6, the above three combinations are exemplarily described with respect to the working tip of FIG. 4 and the handle of FIG. 5.

In FIG. 6, the magnetically-controlled sensor array respectively includes magnetically-controlled sensors A′, B′ and C′. It is assumed that the working tips of different types are represented by the combinations of the magnetically-controlled tips A, B and C.

For the first combination: it is assumed that there are three types of working tips, where the group of magnetically-controlled tips on the working tip the first type include one magnetically-controlled tip A, the group of magnetically-controlled tips on the working tip of the second type include two magnetically-controlled tips A and B, and the group of magnetically-controlled tips on the working tip of the third type include three magnetically-controlled tips A, B and C. In this case, a type correspondence list can be obtained, as shown in Table 1.

TABLE 1

| | First type | Second type | Third type |
|---|---|---|---|
| A | ◯ | ◯ | ◯ |
| B | X | ◯ | ◯ |
| C | X | X | ◯ |

In Table 1, ◯ represents that the corresponding magnetically-controlled tip triggers the corresponding magnetically-controlled sensor to generate an electric identification signal, and x represents that the magnetically-controlled sensor corresponding to the magnetically-controlled tip does not generate an electric identification signal.

For the second combination: it is still assumed that there are three types of working tips and the working tip of each type is represented by two magnetically-controlled tips, specifically as follows: the group of magnetically-controlled tips on the working tip of the first type include two magnetically-controlled tips A and B, the group of magnetically-controlled tips on the working tip of the second type include two magnetically-controlled tips A and C, and the group of magnetically-controlled tips on the working tip of the third type include two magnetically-controlled tips B and C. In this case, a type correspondence list can be obtained, as shown in Table 2.

TABLE 2

| | First type | Second type | Third type |
|---|---|---|---|
| A | ◯ | ◯ | X |
| B | ◯ | X | ◯ |
| C | X | ◯ | ◯ |

In Table 2, ◯ represents that the corresponding magnetically-controlled tip triggers the corresponding magnetically-controlled sensor to generate an electric identification signal, and x represents that the magnetically-controlled sensor corresponding to the magnetically-controlled tip does not generate an electric identification signal. For example, in a sequence corresponding to the first type, the magnetically-controlled tips A and B respectively trigger the magnetically-controlled sensors A′ and B′ to generate a corresponding electric identification signal and then transmit the electric identification signal to the controller 21.

For the third combination: it is assumed that there are totally four types of working tips, which are represented by a combination of the number and the position order, specifically as follows: the group of magnetically-controlled tips on the working tip of the first type include three magnetically-controlled tips A, B and C, the group of magnetically-controlled tips on the working tip of the second type include two magnetically-controlled tips A and C, the group of magnetically-controlled tips on the working tip of the third type include two magnetically-controlled tips B and C, and the group of magnetically-controlled tips on the fourth type of the working tip include two magnetically-controlled tips A and B. In this case, a type correspondence list can be obtained, as shown in Table 3.

TABLE 3

| | First type | Second type | Third type | Fourth type |
|---|---|---|---|---|
| A | ◯ | ◯ | X | ◯ |
| B | ◯ | X | ◯ | ◯ |
| C | ◯ | ◯ | ◯ | X |

In Table 3, ◯ represents that the corresponding magnetically-controlled tip triggers the corresponding magnetically-controlled sensor to generate an electric identification signal, and x represents that the magnetically-controlled sensor corresponding to the magnetically-controlled tip does not generate an electric identification signal.

A process of identifying the type of the working tip according to an electrical identification signal by the controller 21 will be exemplarily described in conjunction with the above three tables.

For Table 1 for the first combination, it is assumed that the type of the currently selected working tip is the second type, and when the working tip and the handle as shown in FIG. 5 are assembled well, the magnetically-controlled sensors A′ and B′ on the handle will be triggered by the magnetically-controlled tips A and B on this working tip to generate an electric identification signal and then transmit the electric identification signal to the controller; and the controller receives the electric identification signal generated by the magnetically-controlled sensors A′ and B′, to know that two magnetically-controlled tips are provided on this working tip according to the correspondence as shown in Table 1, so that the type corresponds to the second type. In this case, a working mode corresponding to the second type can be automatically set according to the result of identification. In this embodiment, the working modes corresponding to the working tips of different types can also be preset, i.e., a correspondence list between the type and the working mode of the working tip can be preset. Moreover, working parameters in the working modes can be preset, and during the setting, an optimal parameter value or a parameter value range is preferably selected for different types by many times of tests or empirically.

For Table 2 for the second combination, it is assumed that the type of the currently selected working tip is the third type, and when the working tip and the handle as shown in FIG. 5 are assembled well, the magnetically-controlled sensors B′ and C′ on the handle will be triggered by the magnetically-controlled tips B and C on this working tip to generate an electric identification signal and then transmit the electric identification signal to the controller; and the controller receives the electric identification signal generated by the magnetically-controlled sensors B′ and C′, to know that two magnetically-controlled tips are provided on this working tip according to the correspondence as shown in Table 2, so that the type corresponds to the third type. And, a working mode corresponding to the third type can be automatically set according to the result of identification.

For Table 3 for the third combination, it is assumed that the type of the currently selected working tip is the first type, and when the working tip and the handle as shown in FIG. 5 are assembled well, the magnetically-controlled sensors A′, B′ and C′ on the handle will be triggered by the magnetically-controlled tips A, B and C on this working tip to generate an electric identification signal and then transmit the electric identification signal to the controller; and the controller receives the electric identification signal generated by the magnetically-controlled sensors A′, B′ and C′, to know that three magnetically-controlled tips are provided on this working tip according to the correspondence as shown in Table 3, and the position order of the magnetically-controlled tips is A, B and C, so that the type corresponds to the first type. And, a working mode corresponding to the first type can be automatically set according to the result of identification.

In this embodiment, in order to improve the identification accuracy, the electric identification signal can also be de-interfered after the electric identification signal generated by the magnetically-controlled sensor is transmitted to the controller and before the type is identified by the controller according to the electric identification signal. A typical method is to use a low pass filter consisting of LCs, a common mode filter, a differential mode filter and a pi filter.

It should be understood that the number of magnetically-controlled sensors in the magnetically-controlled sensor array, the arrangement of magnetically-controlled sensors at the front end of the handle, the combination of the magnetically-controlled tips in the group of magnetically-controlled tips, and the coding rule as shown in Table 1 to Table 3 can all be flexibly set and changed according to the requirements of specific application scenarios. However, implementations are all regarded as falling into the protection scope of the present invention, as long as the type of a currently used working tip is identified by a main controller by arranging different combinations of groups of magnetically-controlled tips on the working tips of different types, generating different combinations of electric identification signals by the magnetically-controlled sensors on the handle and transmitting the electric identification signals to the main controller, as described in the present invention. For example, the number of magnetically-controlled sensors in the magnetically-controlled sensor array can be changed into 4, and the identification of four types of working tips is achieved in a way provided for the first combination. For another example, the number of magnetically-controlled sensors in the magnetically-controlled sensor array can be changed into 2, and the identification of three types of working tips is achieved in a way provided for the third combination. Automatic detection for the type of a working tip is achieved by using the magnetically-controlled tips and the magnetically-controlled sensors arranged on the working tip and the handle, and according to the result of automatic detection, a corresponding working mode is automatically invoked without requiring any manual setting by an operator. Thus, the usage is faster and more convenient. Furthermore, it can avoid the occurrence of unsatisfactory cosmetic results or even medical accidents because the operator forgets setting or sets improperly in an existing manual setting mode, and improve the degree of user satisfaction. Moreover, it can greatly lower the professional requirements on the operator, and is more conducive to product promotion.

In this embodiment, when the type of the working tip is automatically identified in the above way and a corresponding working mode is set to formally enter a working state, and when the working tip during its working is loosened or fallen off due to external factors, after the magnetically-controlled tips on the working tip are separated from the corresponding magnetically-controlled sensors, the magnetically-controlled sensors will generate a signal having a level opposite to that of the electric identification signal; when the signal is detected by the controller, the controller further can stop the working of the beauty apparatus host, or give an alarm to avoid the occurrence of accidents. Thus, the safety performance of the beauty apparatus can be further enhanced, and the degree of user experience satisfaction is improved.

In addition, in order to improve the human-computer interaction experience, with reference to FIG. 7, the beauty apparatus in this embodiment further includes a human-computer interaction console 3 connected to the beauty apparatus host 2. The human-computer interaction console 3 can be configured to issue at least one of a working mode setting instruction and a working state control instruction to the beauty apparatus host, and/or display relevant information from the beauty apparatus host.

Meanwhile, in order to avoid skin damage or even medical accidents to the skin under treatment due to excessively high temperature, in one embodiment of the present invention, a temperature detection module can further be provided on the treating tip, and the temperature detection module is configured to detect a real-time temperature at the skin when the treating tip is in contact with the skin, transmit the temperature to the controller, and judge whether this temperature exceeds a preset temperature threshold during the treatment, and if so, control to stop the beauty apparatus host to avoid the occurrence of the above situation.

The foregoing solution provided by this embodiment is especially applicable to a radio-frequency beauty apparatus. In this case, the treating tip is a radio-frequency treating tip, the host is a radio-frequency host, and the corresponding handle is a handle of the radio-frequency beauty apparatus.

It should be understood that, in the embodiment of the present invention, there is no limitation on the type of the magnetically-controlled sensor, as long as it can be triggered by the magnetically-controlled tip to generate an electric identification signal. Considering that a proximity magnetically-controlled switch can perform detection in a non-contact way, no abrasion and damage will occur. Meanwhile, compared with the output control by mechanical motion, a non-contact output mode is employed, so that it has the advantages of fast response, longer service life and no influence on the life of the contacts, and can guarantee fast and reliable detection. Preferably, the magnetically-controlled sensor can be a proximity magnetically-controlled switch. For example, the proximity magnetically-controlled switch can be a reed switch, a Hall sensor and other proximity magnetically-controlled switches which can achieve the above functions. The present invention will be described by taking a reed switch and a hall sensor as examples.

When the proximity magnetically-controlled switch is a Hall sensor, the Hall sensor can be a common Hall sensor including a Hall element and two electrode wires connected by the Hall element. In this case, the magnetically-controlled tips contain magnetic material, preferably permanent magnetic material; and the magnetically-controlled tips can be completely made of magnetic material, or partially made of magnetic material. For example, the magnetically-controlled tips are made of magnetic conducting material and a magnetic coil, or made of non-magnetic conducting material and a magnetic coil. In the process of the magnetically-controlled tip moving towards the Hall sensor, the Hall element is induced to generate an electrical identification signal and transmit the electrical identification signal to the controller.

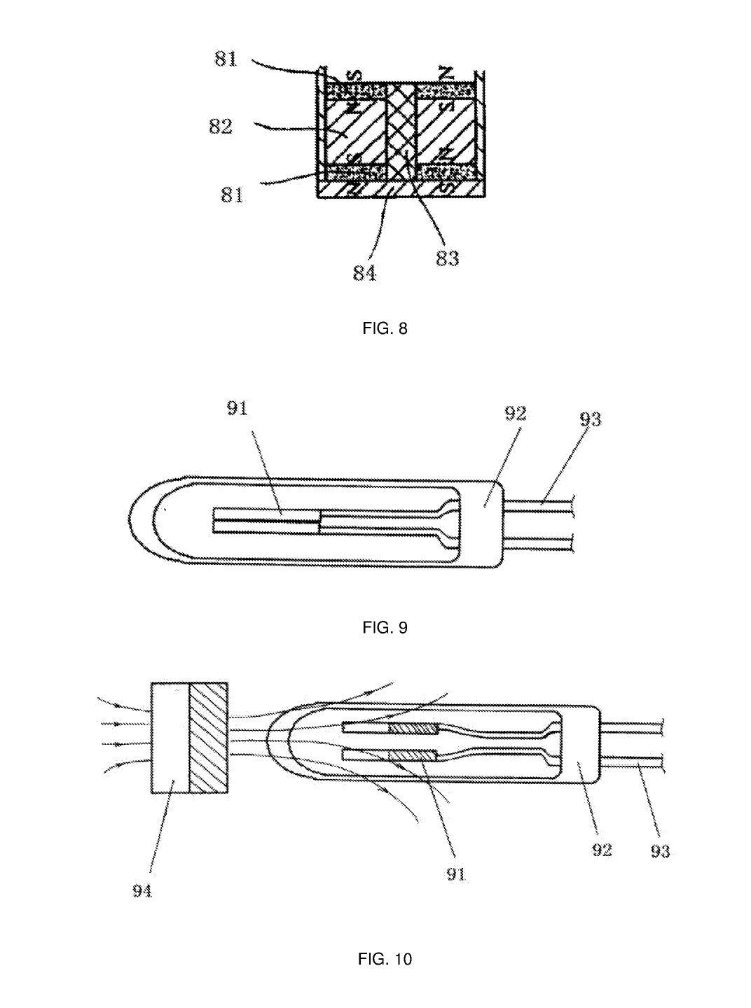

In this embodiment, the Hall sensor itself can also contain a magnet. In this case, the magnetically-controlled tip is made of magnetic conducting material, such as iron. The present invention will be described hereinafter by taking a specific structure as an example. With reference to FIG. 8, the Hall sensor is an embodiment in which two pairs of magnets and one Hall element are used. The Hall sensor includes a shell 84 and a Hall element 83 provided at a front end inside the shell 84. The two pairs of magnets 81 are respectively provided at two ends on the two sides of the Hall element 83, wherein the polarities of the magnets 81 on the same side are the same, with a magnetic conducting block 2 (an iron block can be used) arranged between them. However, the polarities of a pair of opposite magnets 81 on the two sides of the Hall element 83 are opposite. At least the front wall of the shell 84 should be made of non-magnetic conducting material. Of course, the shell 84 can also be completely made of non-magnetic conducting material, to ensure that a magnetic wire can pass through the shell. When the magnetically-controlled tip made of magnetic conducting material moves towards or away from the shell, a magnetic field generated by the magnetic block in the switch will be changed. Therefore, the Hall element is induced to generate an electrical identification signal. The Hall element can also amplify the signal by providing a signal amplification circuit and then output the signal.

When the proximity magnetically-controlled switch is a reed switch, the reed switch can be a normally-closed reed switch or a normally-open reed switch. In this case, the magnetically-controlled tips contain magnetic material, preferably permanent magnetic material; and the magnetically-controlled tips specifically can be completely made of magnetic material, or partially made of magnetic material. The present invention will be further described by respectively taking a specific normally-closed proximity switch and a normally-open reed switch as examples.

With reference to FIG. 9 and FIG. 10, the normally-closed reed switch includes two reeds 91 which are made of ferromagnetic material and packaged into a tubular shell 92, and pins 93 which are connected to the reeds 91 to lead the reeds 91 out from the tubular shell 92. The two reeds 91 are arranged in parallel and in the same direction and are in elastic contact, and the pins 93 are led out from one end of the tubular shell 92. In the absence of an external magnetic field, the two reeds 91 come into contact with each other due to elasticity; at this moment, the reeds are in a closed state; and a high-level signal is generated in a normal state. As shown in FIG. 10, when a magnet 94 is placed at one end of the reed switch (i.e., the magnetically-controlled tip or the front end of the magnetically-controlled tip) so that the magnet 94 is in a longitudinal magnetic field, that is, the direction of a magnetic line is parallel to a magnetic sheet, because the magnetized polarities of the two reeds 91 are the same, the reeds repel and are disconnected from each other to generate a low-level signal, i.e., an electric identification signal.

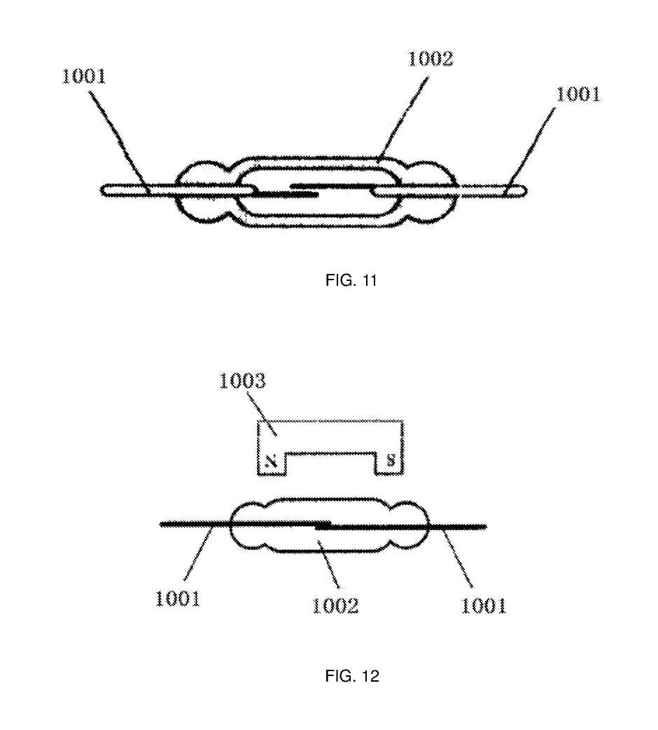

With reference to FIG. 11 and FIG. 12, the normally-open reed switch which consists of a glass tube body 1002 and two pins 1001 has advantages of small volume and convenient control. The normally-open reed switch is coordinated with a permanent magnet 1003 (i.e., the magnetically-controlled tip or the front end of the magnetically-controlled tip), so that a magnetically-controlled switch can be made. The usage is as shown in FIG. 12. In a normal state, the two pins 1001 are separated to generate a low-level signal; the permanent magnet 1003 moves along a direction indicated by an arrow; the two pins 1001 are closed when the permanent magnet 1003 moves towards the reed switch, to generate a high-level signal, i.e., an electric identification signal; and the two pins 1001 are separated when the permanent magnet 1003 moves away from the reed switch.

It should be understood that, in the present invention, the reed switch and the Hall sensor are not limited to several kinds of structures described in the above examples.

In the beauty apparatus according to the present invention, automatic detection for the type of a working tip is achieved by using the magnetically-controlled tips and the magnetically-controlled sensors arranged on the working tip and the handle, and according to the result of automatic detection, a corresponding working mode is automatically invoked without requiring any manual setting by an operator. Thus, the usage is faster and more convenient. Furthermore, it can avoid the occurrence of unsatisfactory cosmetic results or even medical accidents because the operator forgets setting or sets improperly in an existing manual setting mode. Moreover, the falling-off of the working tip during its working can be detected through mutual coordination between the magnetically-controlled tips and the magnetically-controlled sensors arranged on the working tip and the handle, so that the beauty apparatus can be stopped automatically, and its security performance can be further enhanced. Meanwhile, with the advantages of the proximity magnetically-controlled switch, the magnetically-controlled sensor is set as a proximity magnetically-controlled switch to perform detection in a non-contact way, thereby avoiding abrasion and damage, enhancing the response speed and prolonging the service life, and reducing the cost while guaranteeing fast and reliable detection.

The above contents are further detailed descriptions of the present invention with respect to specific implementations, and it is not intended to limit the specific implementations of the present invention to such descriptions. For a person of ordinary skill in the art of the present invention, various simple deductions or replacements may be made without departing from the concept of the present invention, and these deductions or replacements should be regarded as falling into the protection scope of the present invention.

What is claimed is:

1. A working tip of a beauty apparatus, comprising a working tip body, and a treating tip and a group of magnetically-controlled tips respectively provided at front and rear ends of the working tip body, the group of magnetically-controlled tips comprising at least one magnetically-controlled tip; and
when in use, the rear end of the working tip body is assembled and connected to a front end of a handle, and when assembled well, the magnetically-controlled tips in the group of magnetically-controlled tips are respectively coordinated with corresponding magnetically-controlled sensors at the front end of the handle so as to trigger the corresponding magnetically-controlled sensors by way of a magnetic field to generate an electric identification signal.

2. A handle of a beauty apparatus, comprising a handle body, and a magnetically-controlled sensor array having at least two magnetically-controlled sensors which is provided at a front end of the handle body, the magnetically-controlled sensors being connected to a beauty apparatus host; and
when in use, the front end of the handle body is assembled and connected to a rear end of a working tip, and when assembled well, the magnetically-controlled sensors are respectively coordinated with corresponding magnetically-controlled tips in a group of magnetically-controlled tips provided at the rear end of the working tip to generate an electric identification signal and then transmit the electric identification signal to the beauty apparatus host.

3. A hand tool of a beauty apparatus, comprising a handle and at least two working tips of different types; the handle comprises a handle body, and a magnetically-controlled sensor array having at least two magnetically-controlled sensors which is provided at a front end of the handle body, the magnetically-controlled sensors being connected to a beauty apparatus host; the working tip comprises a working tip body, and a treating tip and a group of magnetically-controlled tips respectively provided at the front and rear ends of the working tip body, the group of magnetically-controlled tips comprising at least one magnetically-controlled tip, and combinations of the magnetically-controlled tips in the group of magnetically-controlled tips are different in the working tips of different types; and
when in use, the rear end of the selected working tip body is assembled and connected to the front end of the handle, and when assembled well, the magnetically-controlled tips in the group of magnetically-controlled tips at the rear end of the working tip body are respectively coordinated with corresponding magnetically-controlled sensors at the front end of the handle so as to trigger the corresponding magnetically-controlled sensors to generate an electric identification signal and then transmit the electric identification signal to the beauty apparatus host.

4. A beauty apparatus, comprising a beauty apparatus host and the hand tool according to claim 3; the beauty apparatus host is provided with a controller; and
when in use, the rear end of the selected working tip body is assembled and connected to the front end of the handle, and when assembled well, the magnetically-controlled tips in the group of magnetically-controlled tips at the rear end of the working tip body are respectively coordinated with corresponding magnetically-controlled sensors at the front end of the handle so as to trigger the corresponding magnetically-controlled sensors to generate an electric identification signal and then transmit the electric identification signal to the beauty apparatus host;
the type of a working tip currently mounted on the handle is identified by the controller according to the electric identification signal and a type correspondence list, and a working mode corresponding to the type of the working tip of the beauty apparatus is set according to the result of identification.

5. The beauty apparatus according to claim 4, characterized in that the combinations of the magnetically-controlled tips in the group of magnetically-controlled tips are different in the working tips of different types, comprising: the number of the magnetically-controlled tips is different, and/or the position order for arranging the magnetically-controlled tips are different.

6. The beauty apparatus according to claim 5, characterized in that the type correspondence list comprises a correspondence between various combinations of the magnetically-controlled tips and the type of the working tips; identifying the type of a working tip currently mounted on the hand tool of the beauty apparatus by the controller comprises:
obtaining, by the controller, a combination of magnetically-controlled tips in the group of magnetically-controlled tips on the working tip, according to the received electric identification signal; and
selecting, by the controller and from the type correspondence list, the identified type of the working tip corresponding to the combination of the magnetically-controlled tips.

7. The beauty apparatus according to claim 4, characterized in that the controller is also configured to control the activation of the beauty apparatus host according to the working mode, and then stop the beauty apparatus host when a magnetically-controlled sensor inputting the electric identification signal is detected to input a signal having a level opposite to that of the electric identification signal.

8. The beauty apparatus according to claim 4, characterized in that the beauty apparatus host is a radio-frequency host, and the hand tool is a hand tool of a radio-frequency beauty apparatus.

9. The beauty apparatus according to claim 4, further comprising a human-computer interaction console connected to the beauty apparatus host, configured to issue at least one of a working mode setting instruction and a working state control instruction to the beauty apparatus host, and/or display information from the beauty apparatus host.

10. The beauty apparatus according to claim 4, characterized in that the magnetically-controlled sensor is a proximity magnetically-controlled switch.

11. The beauty apparatus according to claim 10, characterized in that the proximity magnetically-controlled switch is a reed switch or a Hall sensor.

12. The beauty apparatus according to claim 10, characterized in that, when the proximity magnetically-controlled switch is a Hall sensor, the Hall sensor comprises a Hall element and a magnet adjacent to the Hall element, and the magnetically-controlled tips are made of magnetic conducting material.

13. The beauty apparatus according to claim 12, characterized in that, when the proximity magnetically-controlled switch is a reed switch, the reed switch is a normally-closed reed switch or normally-open reed switch, and the magnetically-controlled tips contain magnetic material.

14. The beauty apparatus according to claim 13, characterized in that, when the reed switch is a normally-closed reed switch, the electric identification signal is a low-level signal; and when the reed switch is a normally-open reed switch, the electric identification signal is a high-level signal.
